(19) United States
(12) Plant Patent Application Publication
(10) Pub. No.: US 2002/0174471 P1
(43) Pub. Date: Nov. 21, 2002

(54) Dahlia plant named Dapalipi

(75) Inventor: Francois Van Hee, Emkhuizen (NL)

Correspondence Address: ROCKEY, MILNAMOW & KATZ, LTD., Two Prudential Plaza, Suite 4700, 180 North Stetson, Chicago, IL, 60601, US

(73) Assignee: Ball Floraplant, a division of Ball Horticultural Company

(21) Appl. No.: 09/819,884

(22) Filed: Mar. 28, 2001

(51) Int. Cl. A01H 5/00

(52) U.S. Cl. PLT/321

(57) ABSTRACT

A new and distinct Dahlia plant named Dapalipi characterized by its pale pink double inflorescences, uniform growth habit and dark green foliage.

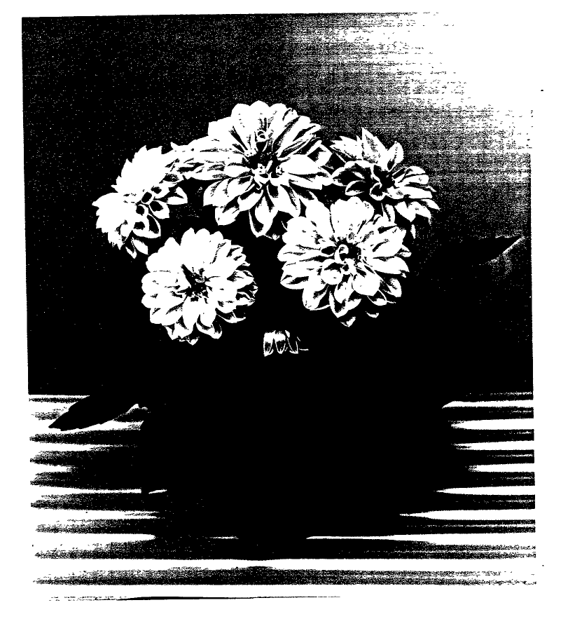

### BACKGROUND OF THE INVENTION

The present invention comprises a new and distinct Dahlia plant botanically known as Dahlia variabilis and hereinafter referred to by the cultivar name Dapalipi. The breeding program which resulted in the production of the new cultivar was carried out in Enkhuizen, The Netherlands. Dapalipi was a naturally occurring mutation of the cultivar Margaret (U.S. Plant Pat. No. 6,769)

Asexual reproduction of the new cultivar by terminal or stem cuttings taken during 2000 at West Chicago Ill., USA has demonstrated that the characteristics of the new cultivar as herein described are firmly fixed and are retained through successive generations of such asexual propagation.

### SUMMARY OF THE INVENTION

It was found that the cultivar of the present invention:

(a) exhibits an upright mounded habit,

(b) forms light pink ray florets and

(c) exhibits a compact to moderate growth habit.

Dapalipi has not been observed under all possible environmental conditions to date. Accordingly, it is possible that the phenotype may vary somewhat with variations in the environment, such as temperature, light intensity, and day length.

The new cultivar of the present invention can be compared to Grace (U.S. Plant Pat. No. 11,709). However in side by side comparison, Dapalipi is more compact, exhibits smaller inflorescences and different ray floret color as detailed in Table A.

### BRIEF DESCRIPTION OF PHOTOGRAPH

The accompanying photograph shows as nearly true as it is reasonably possible to make the same in color illustrations of this type, typical flower and foliage characteristics of the new cultivar. The plant was grown for nine weeks in a greenhouse at West Chicago, Ill., USA.

### DETAILED BOTANICAL DESCRIPTION

The chart used in the identification of colors described herein is the R.H.S Colour Chart of The Royal Horticultural Society, London, England. The color values were determined on Mar. 6, 2001 in West Chicago, Ill. The readings were taken between 10:00 and 11:45 p.m. under natural light conditions. The plants were produced from cuttings taken from stock plants and were grown in a double poly carbonate covered greenhouse under conditions comparable to those used in commercial practice while utilizing a soilless growth medium and maintaining temperatures of approximately 72° F. during the day and approximately 65° F. during the night. Plants were pinched at three weeks after planting. The following measurements and descriptions were taken at eight weeks after planting,

Botanical classification: Dahlia variabilis cultivar Dapalipi

Parentage: Naturally occurring sport or mutation of Margaret

Propagation:

Type cutting.—Terminal tip.

Time to Initiate Roots.—Approximately 7 to 10 days.

Root description.—Fibrous and well branched.

Tubers.—Will form tubers under short day conditions of at least 13 to 14 hours of darkness.

Plant description:

General Appearance and Form.—Upright and mounded, moderately compact.

Height above pot.—Approximately 11.5 cm.

Width.—Approximately 17.6 cm.

Branching habit.—Freely branching. Pinching helps to initiate branching.

Branch description.—Diameter: Approximately 7 mm. Texture: Smooth. Color: 146A. Internode length: Approximately 2 cm.

Foliage description:

Form.—Trifoliate.

Shape.—Ovate.

Margin.—Serrate.

Apex.—Acute.

Base.—Attenuate.

Arrangement.—Opposite.

Venation pattern.—Reticulate.

Surface.—Upper surface slightly rugose.

Color of mature foliage.—Upper surface: Closest to 139A with veins of 146B.

Color of mature foliage.—Lower surface: Closest to 191A with veins of 146B.

Size.—Mature trifoliate leaves are approximately 6.3 cm in length, approximately 4.1 cm in width. Petiole is 4 cm in length, 2.5 mm in diameter and 144A in color.

Flower description:

Outdoor Flowering Habit.—Dapalipi is freely flowering under outdoor growing conditions with substantially continuous blooming from spring through fall.

Flower Appearance.—Double, arising from leaf axils on strong peduncles. Disc and ray florets arranged acropetally on a capitulum.

Inflorescence Diameter.—Approximately 5.5 cm. Disc diameter approximately 1 cm.

Ray florets.—Quantity: Approximately 55. Shape: Elongated, cupped. Length: Approximately 2.2 cm. Apex: Pointed to slightly rounded. Base: Attenuate. Margin: Entire. Texture: Smooth. Color: Between 56D and 56C with base of between 11A and 11B. Florets become lighter toward center of inflorescence.

Disc florets.—Shape: Elongated, cylindrical. Length: 1 cm. Diameter: 2 mm. Color: 11 A.

Involucral bracts.—Quantity: 5. Shape: Diamond-shaped. Tip: Acute. Margin: Entire. Texture: Smooth. Color: 139A.

Reproductive Organs.—Androecium.—On dics florets. Anthers approximately 3 mm in length.

Gynoecium.—On disc and ray florets. Pistil: Approximately 1 cm long. Pollen color is 21A.

Seed production: Seed production has not been observed.

Disease resistance: Disease resistance has not been observed.

TABLE A

| CHARACTERISTIC | DAPALIPI | GRACE |
|---|---|---|
| FULLY OPENED RAY | BETWEEN 56C AND 56D | 155D |
| FLORET COLOR | WITH BASE OF BETWEEN | |
| | 11A AND 11B | |
| FLOWER DIAMETER-CM | 5.5 | 8 |
| PLANT HEIGHT-CM | 11.5 | 17 |

I claim:

1. A new and distinct cultivar of Dahlia named Dapalipi substantially as herein shown and described, which:
(a) exhibits an upright mounded habit,
(b) forms light pink flowers and
(c) exhibits a moderate to compact growth habit.
